(19) United States
(12) Patent Application Publication
(10) Pub. No.: US 2024/0148935 A1
(43) Pub. Date: May 9, 2024

(54) Method For Making An Insertable Medical Device With A Low-Profile Composite Covering Using A Dry Pressing Process

(71) Applicant: Aran Biomedical Teoranta, Co. Galway (IE)

(72) Inventors: David McCarthy, Co. Galway (IE); Tony Durkin, Knocknacarra (IE); Dean King, Knocknacarra (IE); Barry Nugent, Galway City Center (IE)

(21) Appl. No.: 18/539,953

(22) Filed: Dec. 14, 2023

Related U.S. Application Data
(63) Continuation-in-part of application No. 18/142,130, filed on May 2, 2023.
Application No. 18/142,130 is a division of application No. 16/418,182, filed on May 21, 2019, now abandoned.
(60) Provisional application No. 62/674,139, filed on May 21, 2018.

(51) Int. Cl. A61L 27/34; A61F 2/24; A61L 33/06; B29C 43/18

(52) U.S. Cl. CPC A61L 27/34; A61F 2/2409; A61L 33/06; B29C 43/18; A61L 2420/02; B29L 2031/7534

(57) ABSTRACT

A heart valve replacement device and methods of manufacturing same are provided. The heart valve replacement device includes a substrate and a low-profile composite covering in conformal contact with the substrate and suturelessly attached to the substrate. The low-profile composite covering includes a textile base layer and a thermoplastic polymer coating integrated with the textile base layer. The thermoplastic polymer coating or select portions thereof are substantially fluid impermeable.

### RELATED APPLICATIONS

This application a continuation-in-part of U.S. patent application Ser. No. 18/142,130, filed on May 2, 2023, which is a divisional of U.S. patent application Ser. No. 16/418,182, filed on May 21, 2019, now abandoned, which claims the benefit of U.S. Provisional Patent Application Ser. No. 62/674,139, filed May 21, 2018, the entirety of which are hereby incorporated by reference for all purposes.

### TECHNICAL FIELD

The present disclosure relates to insertable medical devices with low profile composite coverings and methods of manufacturing such insertable medical devices.

### BACKGROUND

Blood flow through the human heart is directed through four heart valves: the mitral valve, the tricuspid valve, the aortic valve, and the pulmonary valve. When functioning correctly, these structures act essentially as one-way valves allowing blood to flow forward through the heart, but block blood from flowing backwards. A number of issues can arise within the various heart valves including stenosis (valves not opening sufficiently, typically due to calcification) and regurgitation (the backflow of blood caused by the valve not closing correctly) or a combination of the two, which could necessitate clinical intervention. That intervention may come in the form of surgical replacement of the incompetent heart valve.

Replacement heart valve implants supported by a stent structure can be delivered using catheter-based delivery systems. These prosthetic valves may include expandable stent structures with valve leaflets attached to the inner wall of the stent structure. Replacement heart valves can be crimped down such that it is held on a balloon catheter (e.g., balloon-expandable) or can be contained within the sheath component of a delivery catheter (e.g., self-expanding), and advanced through the vasculature to the target implant site. Once the replacement heart valves is positioned at the target site, the stent structure may be expanded to hold the prosthetic valve firmly in place.

These replacement heart valves are often intended to at least partially block blood flow, and in particular to prevent paravalvular leakage in which blood flows around the valve on the outside of the prosthesis. In order to prevent the occurrence of this issue, replacement heart valves have been developed with skirts covering or partially covering the stent frame structure of the device.

One form of a skirt used for replacement heart valves includes a textile tubular structure constructed by knitting, braiding, weaving or any non-woven textile technique processing yarn fibers into a tubular configuration. Tubular textile structures have the advantage of being naturally porous, which allows desired tissue ingrowth and assimilation into the body. This porosity, which allows for ingrowth of surrounding tissue, is balanced with fluid impermeability to minimize leakage through the body of the skirt. While designing these skirts to minimize permeability it has been necessary to increase the thickness of the textile, providing a highly dense knit or woven construction. In addition, this tight textile construction by its nature reduces the flexibility of the graft material which affects the ability of the stent to which it is attached to crimp and expand unless there is sufficient slack in the textile component. The thickness of the crimped device also impacts the delivery system leading to either an open procedure or a large diameter catheter. A larger catheter can result in a surgeon having fewer options for approaching the target site due to more limited maneuverability.

### SUMMARY

Disclosed herein are insertable medical devices with low profile conformal coverings. In aspect, a heart valve replacement is provided that can comprise a substrate and a low-profile composite covering that is in conformal contact with the substrate and suturelessly attached to the substrate. The low-profile composite covering can include a textile base layer fabricated from a material that provides strength to the low-profile composite covering and a substantially fluid impermeable thermoplastic polymer coating integrated with the textile base layer. The composite covering can cling, conform, and adhere to a complex shaped substrate, can be substantially flush with the substrate, and can conform substantially to the exact shape of the substrate. The composite covering can have a thin wall while remaining substantially fluid impermeable. The composite covering can be heat stabilized into the shape of the substrate and pressed and heated onto the substrate with either soft or granule tooling.

In another aspect, a method of manufacturing a heart valve replacement is provided. Such a method comprises obtaining a substrate of an artificial heart valve, heat stabilizing a textile material, coating the textile material with a thermoplastic polymer, attaching the polymer coated textile material to the substrate to form a composite covering on the substrate, and laminating the composite covering to the substrate.

### BRIEF DESCRIPTION OF THE DRAWINGS

FIG. 1 is a perspective view of a heart valve replacement according to an aspect of the present disclosure.

FIG. 2 is a close-up view of part of a heart valve replacement according to an aspect of the present invention.

FIG. 3 is a block diagram of steps of an exemplary method of manufacturing an insertable medical device according to an aspect of the present disclosure.

FIG. 4 is a block diagram of steps of an exemplary method of laminating a component of an insertable medical device to another component according to an aspect of the present disclosure.

FIG. 5 is a schematic illustration of a compression tool according to an aspect of the present disclosure.

FIG. 6 is a block diagram of steps of an exemplary method of laminating a component of an insertable medical device to another component according to an aspect of the present disclosure.

FIG. 7 is a schematic illustration of a compression tool according to an aspect of the present disclosure.

### DETAILED DESCRIPTION OF THE DRAWINGS

As used herein with respect to a described element, the terms “a,” “an,” and “the” include at least one or more of the described element including combinations thereof unless otherwise indicated. Further, the terms “or” and “and” refer to “and/or” and combinations thereof unless otherwise indicated. By “substantially” is meant that the property or characteristic of the disclosed element need not have the exact described property or characteristic but can have a property or characteristic that is recognizable by one skilled in the art as generally or approximately having the described property or characteristic. Insertable medical devices and components thereof as disclosed herein are used for medical purposes and therefore are sterile. With reference to the composite covering and the substrate of an insertable medical device, “conformal contact” means the composite covering is substantially form-fitting to the substrate such that it conforms to substantially the exact shape of the substrate. A low-profile composite covering has a profile that conforms to the substrate such that the choice of the medical device diameter is not affected or compromised. By “integral” or “integrated” is meant that the described components are not separable using a normal amount of force without damaging the integrity (i.e., tearing) of either of the components. A normal amount of force is the amount of force a user would use to remove a component meant to be separated from another component without damaging either component.

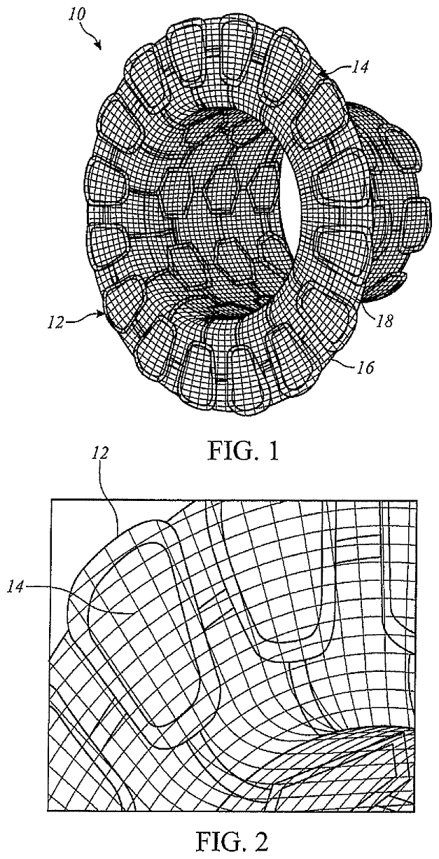

Disclosed herein are insertable medical devices with low profile conformal coverings. Referring to FIG. 1, in an aspect, an insertable medical device 10 comprises a substrate 12, such as a stent frame, and a low-profile composite covering 14. The composite covering can be in conformal contact with the substrate and directly attached to the substrate such that sutures are not required to attach the composite covering to the substrate. This conformal contact is illustrated in FIG. 2, which is a close-up view of the composite covering 14 clinging to and substantially flush with substrate 12. The composite covering can comprise a textile base layer 16 and a thermoplastic polymer coating 18 integrated with the textile base layer 16. Alternatively, the polymer could be coated onto the frame as well as the textile or coated only on the frame. The textile base layer can provide strength to the composite covering whereas the polymer coating can provide fluid impermeability to the composite covering to create a substantially impermeable seal against leaks or backflow of fluid, such as blood. With respect to the latter, a substantially impermeable surface on the substrate or select regions of the substrate can improve medical device functionality such as, for example, minimizing or preventing paravalvular leakage and type I endoleaks. The composite covering can have a low profile, low bulk, high strength construction while maintaining and maximizing the burst strength and/or suture retention strength of the base textile layer. The base textile layer can have larger porosity for added flexibility. Such composite coverings can allow a smaller diameter delivery system to be used to deliver the medical device, which poses less risk of trauma to the patient during insertion of the medical device. In particular, such composite coverings can reduce the overall packing volume of an implantable medical device to allow easier deployment and use of a smaller diameter catheter (e.g., under 30 Fr). Smaller puncture or insertion sites can also result in reduced recovery time for patients. Further, in the case of cardiovascular uses, when crossing through the septum in the heart, the smaller packing volume can allow for a smaller puncture or insertion site, which does not require repair with a secondary implant. This, in turn, allows for follow up surgery through the same insertion site, which a heart patch would preclude. Smaller delivery systems can also allow surgeons to reach more difficult locations, for example, in the peripheral and neurovascular systems of the patient. The low profile of the composite covering can be based on, for example, the thickness of the composite covering, the reduction or elimination of sutures to attach the composite covering to the textile base layer, and reduced creasing during crimping of the medical device onto the delivery device due to the conformity of the thin composite fabric to the frame and the compliance of the fabric. For example, the composite covering can have a thickness of between about 15 μm and about 250 μm.

The substrate can be fabricated from a metallic or polymeric material. It can have a non-tubular shape, a shape different than a coronary stent and typical of a heart replacement valve, and/or a shape with a non-uniform outer diameter. The textile base layer can be fabricated from a biocompatible, high performance, high tenacity (from about 3 to about 100-gram denier) material extruded as either a monofilament or multifilament yarn. Such monofilament or multifilament yarns can be an implantable grade resorbable or non-resorbable polymer material or a mixtures of such materials and yarn materials including, for example, polyesters, including PET polyesters, polypropylenes, polyurethanes, polytetrafluoroethylenes, polyethylenes including ultra-high-molecular-weight polyethylenes, regenerated silk, nylon, liquid crystal polymer, polyether block amide, and suitable combinations thereof. In the case of multifilament yarns, the yarns can be further processed to increase performance through imparting twists into their structure. The textile base layer can be symmetrically attached to the substrate and configured to expand and contract uniformly with the deployment of the medical device. The textile base layer can be seamless and can be fabricated using, for example, circular, weft, double-needle bed warp knitting, weaving, braiding or any non-woven textile technique processing yarn fibers into a tubular configuration. The polymer coating of the composite covering can comprise a thermoplastic polymer such as, for example, thermoplastic polyurethanes, silicone elastomers, polyurethane-silicone co-polymers, polytetrafluoroethylene, fluorinated ethylene/propylene, perfluoroalkoxy fluorocarbon, ethylene/tetrafluoroethylene copolymer and other fluoropolymers, polycarbonate urethanes, polyethylenes, polyamides, polyimides, polyesters, polypropylenes, polyfluoroethylenes, fluorinated polyolefins, fluorinated ethylene copolymer and polyvinylpyr, resorbable polymers such as lactide, glycolide, caprolactone and their co-polymers, polyhydroxybutyrate, polydioxanone, and suitable combinations thereof. The composite covering can be used to bond the composite covering onto the substrate, reducing or eliminating the need for sutures to attach the composite covering to the substrate. The polymer coating can be selectively treated to allow or inhibit tissue integration depending on the desired application of the medical device.

The textile base layer and polymer coating can be a single layer or double layer laminate as described in more detail below. The polymer coating can be integrated with the entire textile base layer or can be integrated with select portions of less than the entire textile base layer such that the textile base layer is selectively uncoated. This may be desired to allow improved tissue ingrowth and integration. The textile fabric layer's porosity, together with an appropriate polymer coating material can be designed to maximize integration of the polymer coating and allow increased areas for lamination in embodiments where the textile fabric layer and polymer coating are a laminate, while minimizing impact on the burst strength and suture retention strength of the medical device. Increased suture retention strength can allow for a more secure attachment of the textile base layer to the target site in the patient's body. The composite covering can be shape-formed to substantially match the implant substrate geometry. The entire composite covering can be porous or non-porous. Alternatively, select portions of the composite covering can be porous or non-porous. For example, select portions of the composite covering can be non-porous to be substantially impermeable to fluid flow, such as blood flow, while other select portions can be porous to promote tissue in-growth. The composite covering can be attached to the entire substrate or can be attached to select portions of less than the entire substrate. For example, as illustrated in FIG. 1, the composite covering 14 can be attached to at least a portion of the outer surface 14a of the substrate. The composite covering can also be attached to at least a portion of the inner surface 14b. The composite covering or the polymer coating can have pharmaceutical agents incorporated therein. For example, a secondary coating process can be used to incorporate pharmaceutical agents into the composite coating. The insertable medical device or components thereof, such as the composite covering or the polymer coating, can also have other types of surfaces such as, for example, an anti-thrombogenic surface, a hydrophilic surface, or a hydrophobic surface.

Non-limiting examples of insertable medical devices include implantable medical devices such as, for example, peripheral, coronary, and neurovascular implantable medical devices. Non-limiting examples of implantable medical device include heart valve replacement and repair implants (e.g., aortic, mitral, tricuspid, and pulmonary); vascular occlusion devices; vascular, (including venous) stents, and grafts; and other types of short or long term or permanent implantable devices. Insertable medical devices, including implantable medical devices, also include gastrointestinal, pulmonary/endobronchial, urinary, and interventional access devices including catheters.

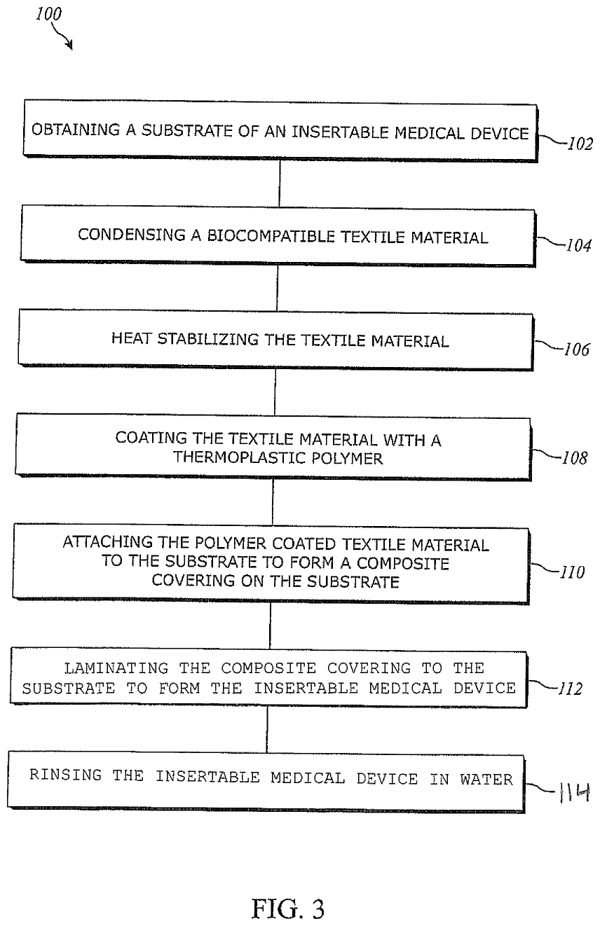

In an aspect, the present disclosure provides methods of manufacturing an insertable medical device. Referring to FIG. 3, a method 100 can include obtaining a substrate, such as a frame having a shape and configuration typical of heart valve replacements, of an insertable medical device 102, optionally condensing a biocompatible textile material 104 and heat stabilizing the textile material 106. Heat stabilization can involve heat treatment of the textile material such that it imparts a pre-determined shape to the textile material. For example, the textile material, constructed to a diameter less than the lowest diameter of a shaped mandrel, can be mounted onto the shaped mandrel such that the soft/greige material of the textile forms around the shape of the mandrel. The mandrel can then be exposed to a heat source, such as placed in an oven, at a temperature above the softening temperature of the textile material. The textile material can then be allowed to cool. Once cooled, if the textile is removed from the mandrel, it will retain the shape of the mandrel, and as such, has been heat stabilized or heat set. Method 100 can further include coating the textile material with a thermoplastic polymer 108. The polymer coated textile material can then be attached to the substrate to form a composite covering on the substrate 110. The polymer coated textile material can be attached to the substrate using a combination of heat and pressure. Prior to attachment, the substrate can be primed or treated to increase the bond strength of the polymer coated textile material to the substrate. Treatments include, for example, surface roughening, use of molecular crosslinkers such as a silane linkage material, use of an intermediate material that bonds more securely to the substrate surface than the textile material and more securely to the polymer coating than the substrate surface. Method 100 can further include laminating the composite covering to the substrate 112 via heat and pressure, for example, to form the insertable or implantable medical device. The lamination process can involve laminating the covering either to the substrate only or, where there are spaces in the structure of the substrate, to itself and around and through the substrate. Selective portions of the substrate can be unlaminated.

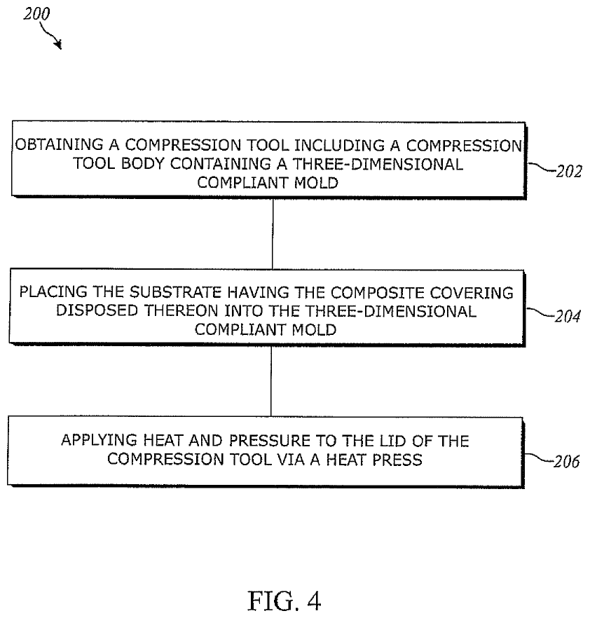

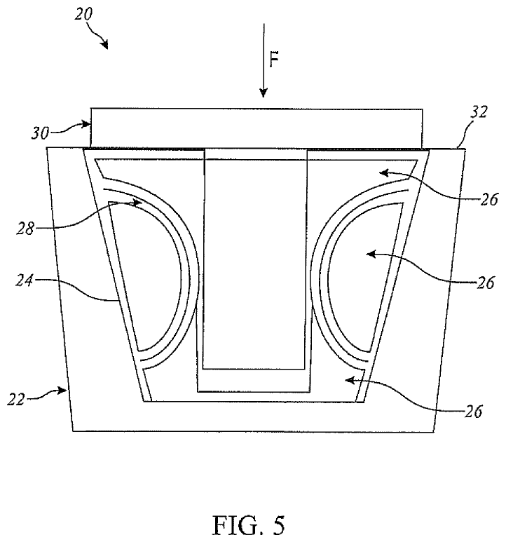

FIGS. 4-7 depict steps and schematically illustrate exemplary lamination processes. Referring to FIGS. 4 and 5, lamination method 200 can comprise obtaining a compression tool (202). As depicted in FIG. 5, a compression tool 20 can comprise compression tool body 22 defining a receptacle 24 including molded three-dimensional compliant tooling members 26 which are shaped to conform to the shape of the substrate. Compression tool 20 can also include lid 30 removably disposed on top surface 32 of compression tool body 22. Method 200 can further include placing the substrate 28 having the composite covering disposed thereon into the three-dimensional compliant mold (204) and applying heat and pressure to the lid via a heat press (206). The pressure and heat distribution within the compression tool can be directed radially using the inserts of the mold 26. Such pressure and heat can translate to the composite covering and substrate to laminate the composite covering to the substrate. The mold inserts 26 can be fabricated from a heat resistant, elastomeric material that is hard enough to exert pressure on the composite covering and the substrate. For example, the mold inserts can be fabricated from silicone or another soft material that does not approach its melting temperature at the temperature required to adhere the composite covering onto the substrate.

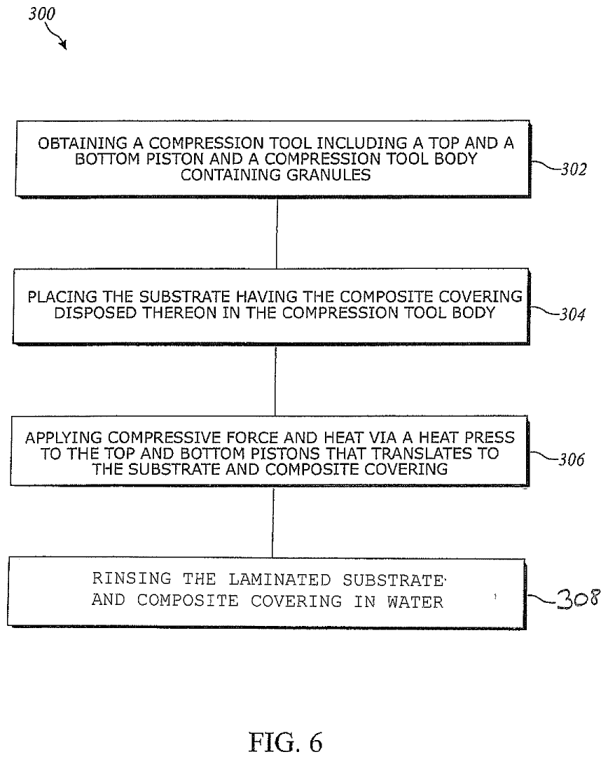

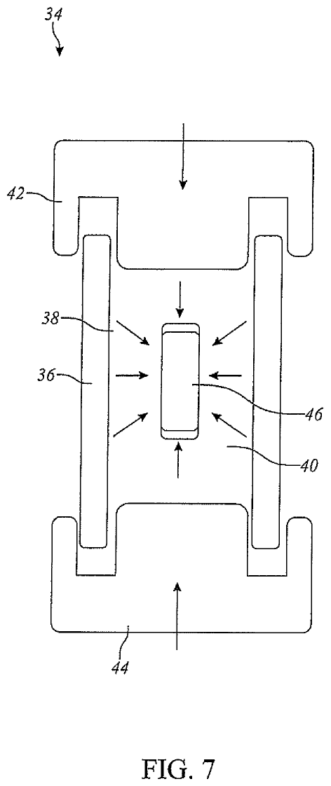

Referring to FIGS. 6 and 7, an alternative method 300 of laminating a composite covering onto a substrate comprises obtaining a compression tool (302). As shown in FIG. 7, a compression tool 34 can comprise a compression tool body 36 defining a receptacle 38 containing a fine, granular, inert, dry medium 40, such as for example, water-soluble sodium chloride or water-soluble calcium chloride. Compression tool 34 can further include top piston 42 slidably coupled to compression tool body 36 and bottom piston 44 slidably coupled to compression tool body 36. Either or both of top and bottom pistons can be removably coupled to the compression tool body. Method 300 can further include placing the substrate having the composite covering disposed thereon 46 into receptacle 38 (304) and applying compressive force and heat via a heat press to the top and bottom pistons (306) that translates through the fine, granular, inert, dry medium 40 to the substrate and the composite covering to laminate the composite covering to the substrate and thereby form an implantable medical device. In particular, medium 40 can impart compressive force and heat to the composite covering to achieve lamination and substrate adhesion. After the composite covering is laminated to the substrate, the at least one of the sodium chloride and calcium chloride is dissolved from the implantable medical device in a water rinse. Sodium chloride and calcium chloride are preferred because they can be finely subdivided into a stable powder, they do not affect the material in which they come in contact with, are biocompatible and non-toxic, and will dissolve away in water, leaving no residue. Rinsing the implantable medical device in water to dissolve the sodium chloride or calcium chloride is shown in step 114 in FIG. 3 and step 308 in FIG. 6.

The heat, both when using a compression tool with granules or a soft mold, comes from the heated press that heats up the outside of the compression tool. The pressure comes from the compressive forces applied to the compression body of the compression tool by the same heated press. Thus, in the molded insert-based compression tool, the molded insert elements transfer the heat and pressure applied to the outside of the compression tool to the inside of the compression tool to the substrate and composite covering. The molded insert elements can be designed to impart pressure in all directions inside the compression tool so that the longitudinal pressure can be redirected radially. Similarly, in the granule-based compression tool, the granules transfer the heat and pressure applied to the outside of the compression tool to the inside of the compression tool to the substrate and composite covering in all directions. Substrates, such as frames, usually cannot be touched by metal tooling, which is the typical material used to perform this kind of heat and pressure transfer in manufacturing of medical devices. Metallic tooling could very easily damage a stent frame so such a material cannot be used. Manufacturing methods as disclosed herein are advantageous in that metal tooling does not directly touch the substrate, such as a stent frame.

Alternatively, the substrate and composite covering can be loaded into a closed vessel and placed in an oven.

Each of the disclosed aspects and embodiments of the present disclosure may be considered individually or in combination with other aspects, embodiments, and variations of the disclosure. Unless otherwise specified, none of the steps of the methods of the present disclosure are confined to any particular order of performance.

What is claimed is:

1. A method for manufacturing an implantable medical device, comprising the steps of:
a) providing a substrate having a plurality of spaces defined by an inner substrate surface spaced from an outer substrate surface, the inner substrate surface defining a lumen extending therethrough;
b) providing a textile fabric layer and heat stabilizing the textile fabric layer;
c) coating the textile fabric layer with a thermoplastic polymer to thereby form a composite covering;
d) obtaining a compression tool, comprising:
i) a compression tool body defining a receptacle, the receptacle including a dry granular, inert medium selected from at least one of sodium chloride and calcium chloride;
ii) a top piston slidably coupled to the compression tool body; and
iii) a bottom piston slidably coupled to the compression tool body;
e) placing the substrate having the composite covering disposed thereon into the receptacle; and
f) applying compressive force and heat to the top and bottom pistons via a heat press that translates through the dry granular, inert medium to laminate the composite covering to the substrate and thereby form the implantable medical device.

2. The method of claim 1, including, after the composite covering is laminated to the substrate, rinsing the implantable medical device in water to dissolve the at least one of the sodium chloride and calcium chloride from the implantable medical device.

3. The method of claim 1, further comprising condensing the textile material prior to coating the textile material with the thermoplastic polymer.

4. The method of claim 3, including providing the top and bottom pistons of the compression tool comprising silicone.

5. The method of claim 1, including, during the heat press, heating the thermoplastic polymer above its glass transition temperature to laminate the composite covering to the substrate.

6. The method of claim 1, including, before placing the substrate having the composite covering disposed thereon into the receptacle of the compression tool, contacting a silane cross-linkage material to the substrate so that the composite covering is attached to the silane cross-linkage material contacted to the substrate.

7. The method of claim 1, including providing the composite covering comprising the thermoplastic polymer coating the textile fabric layer having a total thickness that ranges from about 15 μm and about 250 μm.

8. The method of claim 1, including laminating the composite covering to less than the entire substrate.

9. The method of claim 1, including providing the composite covering having an anti-thrombogenic surface.

10. The method of claim 1, including providing the textile fabric layer comprising a knit, woven, or braided monofilament or multifilament yarn.

11. The method of claim 10, including providing the braided monofilament or multifilament yarn of the textile fabric layer having a tenacity that ranges from about 3-gram denier to about 10-gram denier.

12. The method of claim 1, including fabricating the substrate from a metallic or a polymeric material.

13. The method of claim 1, including selecting the textile fabric layer from an implantable grade resorbable polymer yarn, a non-resorbable polymer yarn, and a mixture of resorbable and non-resorbable yarns.

14. The method of claim 10, including extruding the monofilament or multifilament yarn from a PET polyester, a polypropylene, a polyurethane, a polytetrafluoroethylene, an ultra-high-molecular-weight polyethylene, a regenerated silk, a nylon, a liquid crystal polymer, a polyether block amide, and combinations thereof.

15. The method of claim 10, including twisting the multifilament yarn.

16. The method of claim 1, including selecting the thermoplastic polymer of the composite covering from a thermoplastic polyurethane, a silicone elastomer, a polyurethane-silicone co-polymer, a polytetrafluoroethylene, a fluorinated ethylene/propylene, a perfluoroalkoxy fluorocarbon, an ethylene/tetrafluoroethylene copolymer, a polycarbonate urethane, a polyethylene, a polyamide, a polyimide, a polyester, a polypropylene, a polyfluoroethylene, a fluorinated polyolefin, a fluorinated ethylene copolymer, a polyvinylpyrrolidone, a lactide, glycolide, a caprolactone, a polyhydroxybutyrate, a polydioxanone, and combinations thereof.

17. The method of claim 1, including roughening the substrate before laminating the composite covering to the substrate.

18. The method of claim 1, including selectively leaving a portion of the textile fabric layer uncoated with the thermoplastic polymer.

19. The method of claim 1, including providing the substrate having a shape that is typical of a heart valve repair or replacement device.

20. The method of claim 1, including providing the compression tool comprising a three-dimensional compliant mold shaped to conform to a shape of the substrate.
